(19) United States
(12) Patent Application Publication
(10) Pub. No.: US 2010/0239673 A1
(43) Pub. Date: Sep. 23, 2010

(54) Blood compatible nanomaterials and methods of making and using the same

(76) Inventors: Robert J. Linhardt, Albany, NY (US); Saravanababu Murugesan, Troy, NY (US); TaeJoon Park, Troy, NY (US)

Correspondence Address: ELMORE PATENT LAW GROUP, PC, 515 Groton Road, Unit 1R, Westford, MA, 01886, US

(21) Appl. No.: 11/497,838

(22) Filed: Aug. 1, 2006

Related U.S. Application Data
(60) Provisional application No. 60/704,383, filed on Aug. 1, 2005.
(60) Provisional application No. 60/704,384, filed on Aug. 1, 2005.

(51) Int. Cl. A61K 9/14; C07H 3/00; C08B 37/10; A61K 31/727; A61K 31/715; C07K 14/00; A61P 13/12

(52) U.S. Cl. 424/488; 536/1.11; 536/118; 536/123.1; 514/56; 514/54; 530/395; 977/752; 977/750

(57) ABSTRACT

The invention provides blood compatible nanomaterials, biomaterials prepared therewith and blood compatible medical devices fabricated using the biomaterials of the invention. The invention further provides methods of making and using the nanomaterials, biomaterials and medical devices of the invention for the diagnosis, prevention and treatment of medical conditions. The invention further provides methods of using room temperature ionic liquids to make blood compatible nanomaterials.

### RELATED APPLICATIONS

This application claims the benefit of U.S. Provisional Application No's. 60/704,383 and 60/704,384 both filed on Aug. 1, 2005. The entire teachings of the above applications are incorporated herein by reference.

### GOVERNMENT SUPPORT

The invention was supported, in whole, or in part, by NIH grant number HL52622. The Government has certain rights in the invention.

### BACKGROUND OF THE INVENTION

The ability of medical scientists to diagnose, treat and repair diseased and damaged tissues has increased dramatically in recent years. As new diagnostic and treatment devices are developed, medical scientists seek the optimum material for each application. The target anatomical site and intended use dictate the physical qualities demanded from candidate materials. Just as the human body has evolved into a variety of different tissue types, each perfectly adapted for its role, medical devices must be composed of equally specialized materials. For example, in vivo medical devices including catheters, and probes designed for insertion into narrow body structures such as the urethra, arteries, veins, and spinal column must have a minimal diameter, extreme flexibility, resilience and durability. Prosthetic medical devices such as artificial hips and joint replacements must be rigid and capable of surviving severe impact. Extracorporeal devices such as heart-lung machines and kidney dialysis equipment are complex mechanical devices that demand a diversity of functional and structural materials, each optimized for a particular function which may include contact with human tissues and body fluids.

In spite of the ongoing success of such devices, extracorporeal, in vivo, and prosthetic medical devices necessarily have surfaces that come into direct contact with blood and/or other body fluids and tissues, it is essential that the surfaces of these medical device be biocompatible. Thus, such biocompatible surfaces should not stimulate blood clotting (thrombogenesis), induce inflammatory or immune responses, kill or damage host tissues, or release toxic compounds when in contact with blood or living tissues. Of these biocompatibility issues, blood compatibility is the most significant. The blood compatibility issue associated with the surfaces of materials commonly used to produce medical devices is their natural propensity to induce thrombogenesis. When this occurs on the surface of an implanted medical device, or within the chambers of an extracorporeal device, there is a potential risk of thromboembolism—the blocking of a blood vessel by a particle that has broken away from a blood clot—possibly resulting in a heart attack, lung failure, or stroke. Therefore, it has been, and continues to be, a primary focus of materials scientists and biomedical engineers to enhance the blood compatibility of a medical device and to reduce or eliminate the thrombogenic potentials associated with the materials commonly used in medical device manufacturing.

At present, the most successful techniques known in the art for reducing thrombogenesis have evolved from the observation that certain compounds, when administered systemically, prevent blood clot formation. The most commonly used of these therapeutic anticoagulants is heparin, an acidic mucopolysaccharide that acts in conjunction with naturally occurring antithrombin III to inhibit most of the serine proteases in the blood coagulation pathways. However, the use of systemic anticoagulants is not without risks. Heparin, for example, is metabolized through the liver and normally a single therapeutic dose will continue to inhibit blood clot formation in the patient for several hours. Should a traumatic event occur during the time systemic heparin is at therapeutic levels, the patent's ability to control bleeding will be impaired. Therefore, in an effort to reduce the sometimes potentially lethal side effects associated with systemic anticoagulants used in conjunction with medical devices, and to increase surface biocompatibility, materials scientists have experimented with anti-thrombogenic coatings that are intended to inhibit clot formation at its source, rather than systemically.

The design of blood compatible macroscopic devices has primarily relied on making their surfaces resemble the luminal surface of the endothelium through the immobilization of the glycosaminoglycan (GAG), heparin. Proteoglycans are macromolecules that consist of a core protein to which multiple GAG chains are linked. Endothelial heparan sulfate proteoglycan regulates the coagulation cascade, while other PGs play essential physiological, biochemical and structural roles within all animals and are involved in the compression resistance, arresting the movement of microorganisms, signal transduction and cytoskeletal support. The GAG chains are primarily responsible for the functional aspects of proteoglycans and are well studied and like heparin, are widely used pharmaceuticals. Functional studies and applications of intact proteoglycans are less common because of their limited availability and the susceptibility of their core proteins to proteolysis and denaturation. These problems have largely precluded the use of proteoglycans as therapeutic agents and in biomaterials. It would be desirable to provide stable proteoglycans for use as therapeutic agents and in biomaterials.

Carbon nanomaterials and particularly carbon nanotubes represent one of the most widely used building blocks for nanodevices and have also been successfully used as solid supports for biofunctionalization. Carbon nanotubes, with their unique structural, electronic and mechanical properties have an enormous number of applications in making various materials including nanotube polymeric composites, electronic and optical devices and enzyme/catalytic supports. Nanotubes are often preferred over metallic or non-metallic nanoparticles for biomedical applications because of their larger inner volume, distinct inner and outer surfaces and open mouths. These properties enable the filling of nanotubes with desired species (small molecule or macromolecule), differential modification of inner and outer surfaces and access to inner surfaces for incorporation of species.

It would be desirable to exploit the advantages of nanotechnology to provide superior blood-compatible biomaterials for use in medicine.

### SUMMARY OF THE INVENTION

The invention provides blood compatible nanomaterials, biomaterials prepared therewith and blood compatible medical devices fabricated using the biomaterials of the invention. The invention further provides methods of making and using the biomaterials and medical devices of the invention for the diagnosis, prevention and treatment of medical conditions. The invention further provides methods of using room temperature ionic liquids to make blood compatible nanomaterials.

### BRIEF DESCRIPTION OF THE DRAWINGS

FIG. 1 a-f are micrographs showing tapping mode (TM) AFM, topography (a,c,e) and phase (b,d,f) images of pristine (a,b) MWNTs, PEI coated (c,d) MWNTs and heparinized (e,f) MWNTs. Pristine and PEI coated MWNTs show uniform diameters (a and c) while heparinized MWNTs showed variable diameters. The smooth surfaces of the pristine MWNTs and PEI coated MWNTs are contrasted to the rough, uneven surface with bulges of the heparinized MWNTs associated with domains of immobilized heparin (bold arrows in f).

FIG. 2 is a cartoon comparing blood compatibility of a carbon nanotube coated with heparin in accordance with the invention as compared to a pristine (unmodified) carbon nanotube.

FIG. 3 is a schematic representation for the preparation of composite biomaterials of the invention.

FIG. 4a-f is a micrograph showing the surface morphology of the cellulose and the heparin-cellulose composite films. FESEM (a,b); AFM topography (c,d); and AFM phase (e,f) images of the cellulose only film (a,c,e) and heparin-cellulose composite film (b,d,f). FESEM images are presented at 30,000× magnification.

FIG. 5a-b are field emission scanning electron microscope images of cellulose only fibers, 10% (w/w).

FIG. 6a-b are field emission scanning electron microscope images of cellulose-heparin composite fibers (10% (w/w) cellulose solution, 7% heparin-cellulose final concentration).

### DETAILED DESCRIPTION OF THE INVENTION

As used herein the term “nanomaterial” refers to a nanoscale (approximately 1-100 nm in at least one dimension). Nanomaterials in accordance with the invention may be in the form of a hollow sphere, full sphere, ellipsoid, fiber, wire, pyramid, prism or tube. Cylindrical nanomaterials are sometimes referred to herein as, “nanotubes” or “nanofibers”. Nanotubes may be single-walled nanotubes (SWNTs), double-walled nanotubes (DWNTs) or multi-walled nanotubes (MWNT). Most SWNTs have a diameter of close to 1 nm, with a tube length that can be many thousands of times larger. Nanotubes can be mass-produced through arc-discharge, laser vaporization, plasma enhanced chemical vapor deposition, thermal chemical vapor deposition, vapor phase growth, electrolysis, flame synthesis, and the like.

As used herein the term “neoglycoprotein” refers to a synthetic glycoprotein comprising at least one polysaccharide linked to a nanomaterial core which replaces a protein core. As used herein the term “neoproteoglycan” refers to a synthetic proteoglycan comprising at least one glycosaminoglycan linked to a nanomaterial core instead of a protein core. As used herein, the phrase “linked to a nanomaterial core” is meant to include covalent and non-covalent molecular interactions between the polysaccharide and the core. Replacement of the core proteins of proteoglycans with nanomaterial cores in accordance with the invention avoids the problems of proteolysis and denaturation associated with attempts to use intact proteoglycans as therapeutics and in biomaterials. The neoglycoproteins and in particular, the neoproteoglycans of the invention are highly stable and suitable for use as therapeutics and in biomaterials, particularly blood compatible biomaterials.

As used herein the term “composite” means a material comprising two or more distinct components in one or more phases. Composites of the invention may take various forms and shapes, for example, rigid and flexible tubes, sheets, membranes, fibers, or formed and contoured shapes. Composites of the invention may be nanosized materials, micro-sized materials or larger (macro) sized materials.

As used herein, the term “fibers” includes “nano-fibers”, “micro-fibers” or larger-sized fibers and refers to fibers comprising branched or unbranched cylindrical structure comprising carbon, polymeric, non-polymeric, or block polymeric compounds, metallic compounds, non-metallic compounds or composites.

As used herein a “biomaterial” means any synthetic, natural or modified natural material or any combination thereof that is used in contact with biological systems and which are biocompatible therewith. Although biomaterials of the invention are primarily used for medical applications, they may also be used to grow cells in culture, to assay for blood proteins in the clinical laboratory, in processing biomolecules in biotechnology, and a myriad of other uses involving the interaction between biological systems and synthetic, natural or modified natural materials. The biomaterials of the invention may take various forms and shapes, for example, rigid and flexible tubes, sheets, membranes, fibers, or formed and contoured shapes for use in medical equipment or in patients as implants. Such biomaterials may be used in the design, production, manufacture and fabrication of a number of medical devices.

As used herein the term “biocompatible” refers to the ability of a biomaterial to perform its intended function while in contact with the appropriate biological system without inducing adverse effects in or to the biological system in which it is associated.

As used herein, the term “medical device” is any instrument, apparatus, implement, machine, contrivance, implant, in vitro reagent, or other similar or related article, including a component part or accessory, which is intended for use in the diagnosis and imaging of disease or other conditions, or in the cure, mitigation, treatment, or prevention of disease in humans or animals. Medical devices include but are not limited to: vascular grafts; catheters; heart valves; cardiovascular stents; breast implants; implantable tissue scaffolds; pacemakers; renal dialyzers; left ventricular assist devices; joint prosthesis; prosthetic organs; artificial blood vessels; intraocular lens; and dental implants. Medical devices include intravascular medical devices such as synthetic (prosthetic) grafts, implantable pumps, heart valves and stents adapted for long term or permanent insertion into the lumen of a blood vessel, e.g., in conjunction with percutaneous transluminal angioplasty. Medical devices also include intravascular devices adapted for temporary insertion in a blood vessel, e.g., a balloon or catheter tip. Medical devices also include extravascular medical devices, such as plastic tubing or a membrane insert in the extravascular path of the blood stream of a living being undergoing a medical procedure requiring the cycling of the blood stream or a portion thereof outside the body of the living being, e.g., coronary artery bypass surgery or renal dialysis. Medical devices also include drug delivery devices (e.g. drug delivery patches), gene delivery devices, and cell growth matrices.

As used herein the term “biomimetic” refers to biomaterials having properties that are similar to or “mimic” the properties of compositions made by living organisms. The neoproteoglycans of the present invention are biomimetic in that they have properties similar to naturally occurring proteoglycans and provide the same or similar physiological functions in the body or in extracorporeal settings as would naturally occurring proteoglycans. Biomimetic biomaterials of the invention are particularly well suited for fabrication of hemodialysis (e.g. artificial kidney, dialysis membranes) in accordance with the present invention.

As used herein the term “blood compatible” when used in reference to the biomaterials and medical devices of the invention means that such materials or devices are capable of regulating blood coagulation when in contact with blood without inducing adverse effects to the system in which it is present. Such adverse effects that are avoided by the present invention include adsorption of plasma proteins, platelet adhesion and activation, triggering the coagulation and complement cascade and clot formation. As used herein “regulating blood coagulation” includes the diminution or prevention of undesired blood coagulation and adverse effects as compared to prior art materials and devices and may optionally include enhancing or improving blood flow of the medical device including increasing plasma recalcification time and activated partial thromboplastin time (APTT) and enhancing anticoagulant properties.

The terms “ionic liquid” (“IL”) and “Room Temperature Ionic Liquid” (“RTIL”) are used interchangeably herein and refer to a liquid that is composed almost entirely of ions. The ionic liquid may optionally contain from 0 up to about 15, preferably less than 10% by volume of water. Ionic liquids are organic compounds that are liquid at room temperature. They differ from most salts, in that they have very low melting points. They tend to be liquid over a wide temperature range and have essentially no vapor pressure. Most ILs are air and water stable. ILs remains a liquid at room temperature or below (even as low as −100° C.). Such ILs may be designed to have a liquid range of 300° C. which is larger than that of water. ILs are generating increased interest as “green” solvents, because of their low vapor pressure and recycling possibility. RTILs that are capable of dissolving many non-polar and polar compounds including carbohydrates have been designed and synthesized. Murugesan et al., Synlett, 2003; 9:1283-1286 and Murugesan et al., Curr. Org. Synth. 2005; 2:437-451.

The term “electrospinning” refers to a process to make microfibers or nanofibers from a polymer solution through electrostatic force.

The compositions and methods of the invention provide a means for inhibiting platelet aggregation and platelet adhesion that trigger the coagulation complement cascade, which often manifests in the form of a layer that builds up on a medical device that is permanently implanted in a blood vessel or that comes in contact with the circulating blood of a living being on a temporary basis (including extracorporeal synthetic circuits for applications such as cardiopulmonary bypass or kidney dialysis) or in the form of a detachable clot which, if it travels to the organs such as brain, lung, heart, kidney and liver, can be debilitating or life-threatening. The compositions of the invention further provide enhanced blood compatibility of biomaterials and medical devices fabricated using the compositions of the invention.

In one aspect, the invention provides blood compatible nanomaterials. The nanomaterials may be comprised carbon, polymers and/or block polymers, non-polymeric compounds, metallic or non-metallic compounds or a composite material. The blood compatible nanomaterials of the invention may be any nanoshape including but not limited to, hollow or solid spheres, ellipsoids, fibers, wires, pyramids, prisms, tubes or any combination thereof. In one preferred embodiment, the nanomaterials are in the shape of nanotubes and may be SWNTs, DWNTs or MWNTs.

Examples of polymers useful as blood compatible nanomaterials include but are not limited to carbohydrates, polysaccharides, proteins, oligonucleotides, polyamides, polycarbonates, polyalkenes, polyvinyl ethers, polyglycolides, and polyurethanes. Examples of block polymers useful as nanomaterials include but are not limited to, block copolymers comprising one or more polyolefin blocks, one or more vinyl blocks or one or more methacrylate blocks. Examples of block copolymers useful as blood compatible nanomaterials of the invention include, but are not limited to, polyethylene oxide (PEO), and silicone-vinyl block polymers. Examples of non-polymeric compounds include useful as nanomaterials include, but are not limited to glass, silicon, boron nitride, tungsten sulfide, and boron hydrides.

Examples of metallic compounds include but are not limited to, gold, silver, iron, metallo-carbohedranes (“met-cars”), and titanium. Examples of non-metallic compounds include, but are not limited to, metal derivatives such as oxides, and sulfides including titania, zirconia, cerium oxide, zinc oxide, and iron oxide. In one preferred embodiment, the nanomaterial comprises carbon.

In one embodiment, the blood compatible nanomaterials of the invention are neoglycoproteins and preferably, neoproteoglycans comprising nanomaterial cores to which polysaccharides such as glycosaminoglycans are covalently and/or non-covalently linked. Replacing the susceptible protein cores of the glycoproteins and proteoglycans with a nanomaterial in accordance with the invention yields highly stable compounds having features and properties that resemble protein-containing glycoproteins and proteoglycans. Such features and properties include those of, for example, heparan sulfate which regulates the blood coagulation cascade. Neoproteoglycans of the invention are particularly useful in the preparation of composites, biomaterials, blood compatible materials, and biomimetic materials in accordance with the invention.

In one embodiment, carbon nanotubes including SWNTs and MWNTs comprise the cores of the neoproteoglycans of the invention. In one embodiment, neoproteoglycans of the invention comprise at least one glycosaminoglycan selected from heparan sulfate, heparin, dermatan sulfate, keratan sulfate, hyaluronate, chondroitin sulfate, hybrids thereof, derivatives thereof or any combination thereof.

Polysaccharides may be covalently or non-covalently linked to a nanomaterial core using several approaches including, for example, physical adsorption via hydrophobic interaction of a coating on the nanotubes, covalent linking between the oxidized nanotubes and a reactive moiety of the polysaccharide or any combination of physical adsorption and covalent linking including covalent linking of the polysaccharide to a coating physically adsorbed on the nanomaterials.

In one embodiment, the invention provides methods of making neoproteoglycans comprising the steps of: (a) coating a carbon nanomaterial with a reactive compound; (b) activating at least one glycosaminoglycan; and (c) coupling the activated glycosaminoglycan with the coated nanomaterial of step (a). In one embodiment, the reactive compound of step (a) comprises a nitrogen-containing reactive moiety or a hydroxyl function. The reactive compound may be covalently or non-covalently linked to the carbon nanotube. Examples of reactive compounds suitable for coating the carbon nanomaterial include, but are not limited to: polyethyleneimine (PEI), polyallylamine and polyethylene oxide. The coupling of step (c) includes attachment to the nanotube via a covalent and/or non-covalent linker.

In one embodiment, the invention provides methods of making neoproteoglycans comprising the steps of functionalizing a carbon nanotube, by, for example, oxidation to form a free carboxyl moiety, and covalently linking an activated glycosaminoglycan to the functionalized nanotube.

In one aspect, the invention provides blood compatible nanomaterials comprising a composite. In one embodiment, the composite comprises a modified or unmodified carbohydrate matrix having an agent homogenously distributed therein. The agent may be an anticoagulant, antithrombogenic, antibiotic, diagnostic agent, imaging agent, fluorescent agent, radioactive agent or anticancer agent.

Modified or unmodified carbohydrates suitable for use as a matrix include but are not limited to, cellulose, starch, hemicellulose, inulin, chitin, chitosan, glycogen, pectin, carageenan, fucan, fucoidin, derivatives of the foregoing, hybrids of the foregoing or any combinations thereof. An example of a modified carbohydrate or carbohydrate derivative is cellulose acetate. An example of a hybrid is a cellulose-chitin composite.

In one preferred embodiment, the invention provides a composite comprising heparin homogenously dispersed within a cellulose or modified cellulose matrix. Cellulose, a carbohydrate polymer, has very good thermal, mechanical, bio-stable and biocompatible properties. Chemically modified celluloses find a wide range of medical applications, particularly in the preparation of dialysis membranes and implantable sponges. The chemical modification of cellulose is complicated by its high degree of crystallinity resulting in its insolubility in water and most conventional organic solvents (other than N-methylmorpholine-N-oxide, CdO/ethylenediamine, LiCl/N,N′-dimethylacetamide and near supercritical water).

Heparin has also proven to be difficult to work with in the past. Up until just recently, up to 10 mg of heparin was soluble only in dimethyl sulfoxide, dimethyl formamide and formamide apart from water. The present inventors have recently reported the dissolution of up to 10 mg/ml heparin in the RTILs 1-ethyl, 3-methylimidazolium benzoate ([emIm][ba]) and 1-butyl, 3-methylimidazolium benzoate ([bmIm][ba]). Marugesan et al., Carbohydr. Polym. 2006; 63; 268-271.

In one aspect, the invention provides methods of making a composite comprising a carbohydrate matrix having at least one agent homogenously distributed therein comprising the steps of combining a solution of a carbohydrate dissolved in an ionic liquid with one or more agents also dissolved in one or more ionic liquids and mixing under conditions and temperatures in which the matrix and the one or more agents are stable to form a combined solution comprising the carbohydrate and one or more agents dispersed therein. The combined solution may then be fabricated in the form of a composite material before or after the removal of any residual ionic liquid. In one preferred embodiment, at least two of the ionic liquids used in the method are not the same.

The properties of the ionic liquids suitable for use in the present invention can be tailored by varying the cation and anion comprising the IL. Examples of ionic liquids are described, for example, in J. Chem. Tech. Biotechnol., 68:351 356 (1997); Chem. Ind., 68:249 263 (1996); and J. Phys. Condensed Matter, 5:(supp. 34B):B99 B106 (1993), Chemical and Engineering News, Mar. 30, 1998, 32 37; J. Mater. Chem., 8:2627 2636 (1998), and Chem. Rev., 99:2071 2084 (1999), the contents of which are hereby incorporated by reference. Ionic liquids are also described, for example, in U.S. Pat. Nos. 6,808,557 and 6,824,599 incorporated herein by reference. Other ionic liquids will be apparent to those skilled in the art.

Many ionic liquids are formed by reacting a nitrogen-containing heterocyclic ring, preferably a heteroaromatic ring, with an alkylating agent (for example, an alkyl halide) to form a quaternary ammonium salt, and performing ion exchange or other suitable reactions with various counter ions such as Lewis acids or their conjugate bases to form ionic liquids (nitrogen based ionic liquid). Examples of suitable heteroaromatic rings include substituted pyridines, imidazole, substituted imidazoles, pyrrole and substituted pyrroles. These rings can be alkylated with virtually any straight, branched or cyclic C₁₋₂₀ alkyl group, but preferably, the alkyl groups are C₁₋₁₆ groups, since groups larger than this tend to increase the melting point of the salt. Ionic liquids have also been based upon various triarylphosphines, thioethers, and cyclic and non-cyclic quaternary ammonium salts.

Counterions which have been used include chloroaluminates, bromoaluminates, gallium chloride, tetrafluoroborate, tetrachloroborate, hexafluorophosphate, nitrate, trifluoromethane sulfonate, methylsulfonate, p-toluenesulfonate, hexa fluoroantimonate, hexa fluoroarsenate, tetrachloroaluminate, tetrabromoaluminate, perchlorate, hydroxide anion, copper dichloride anion, iron trichloride anion, zinc trichloride anion, as well as various lanthanum, potassium, lithium, nickel, cobalt, manganese, and other metal-containing anions. One preferred anion comprises benzoate.

Preferred ionic liquids of the invention include 1-butyl, 3-methylimidazolium chloride [bmIm][Cl], 1-ethyl, 3-methylimidazolium benzoate ([emIm][ba]), 1-butyl, 3-methylimidazolium benzoate ([bmIm][ba]), and 1-butyl, 3-methylimidazolium hexa fluorophosphates [bmIm][PF₆].

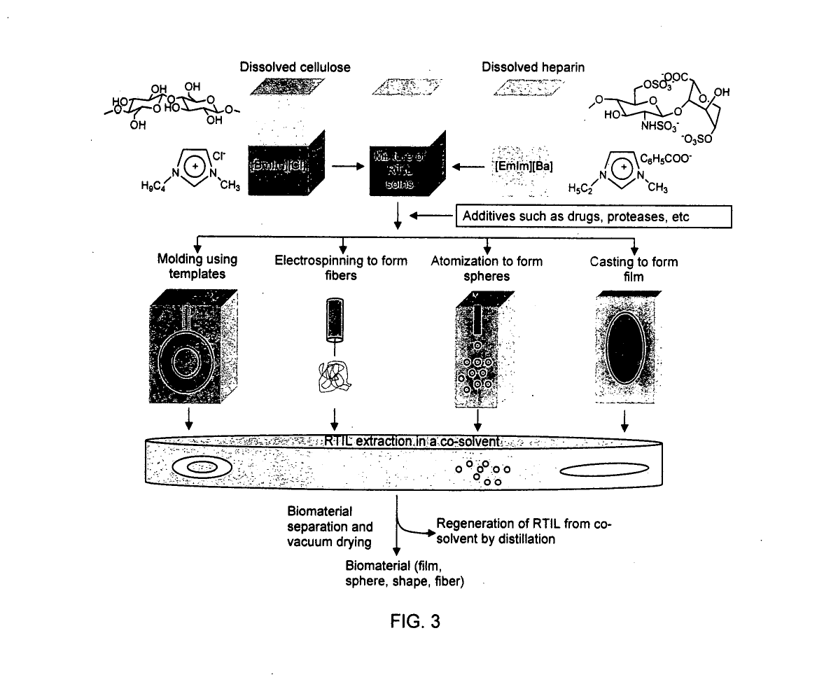

In accordance with the methods of the invention, the combined ionic liquid solution comprising the carbohydrate matrix and the one or more agents may be fabricated into various shapes and forms to form composite materials and nanomaterials. Techniques used to form composite materials and nanomaterials are shown in FIG. 3 and include but are not limited to molding and casting as is known in the art to form films or other desired shapes, electrospinning to form nanofibers and microfibers, or atomizing to form spheres. Other methods for preparing composite materials and nanomaterials from solutions are well known to those skilled in the art.

While the nanomaterials of the invention may comprise composites, the compositions and methods described herein for preparing nanocomposites are also suitable for preparing larger materials including microcomposites and macrocomposites.

In one preferred embodiment, a composite of the invention is formed into nanofibers, microfibers or combinations thereof, to form biomaterial that may be woven into the form of a membrane suitable for use for kidney dialysis. Kidney dialysis membranes in accordance with the invention exhibit superior clotting kinetics and allow the passage of urea while retaining albumin.

In accordance with the invention, some or all of the residual IL may be removed prior to, during or after the solidification step of the composite. The residual IL may be removed by washing with a cosolvent or extracting with a cosolvent. Suitable cosolvents include but are not limited to ethanol, isopropanol, methanol, water, hexanes, ethyl acetate, acetonitrile or any other volatile solvent that does not dissolve both the matrix and the agent. Optionally, the ionic liquid may be regenerated from the cosolvent for reuse by distillation.

The nanomaterials of the invention are useful in the design, and fabrication of biomaterials, biomimetic materials, blood compatible materials, and medical devices as described herein.

In one embodiment, the invention provides a solution comprising a mixture of a matrix material dissolved in a first ionic liquid in combination with at least one agent dissolved in at least one different ionic liquid wherein the matrix and the agent remain stable in the solution mixture. Although ionic liquids have been known to provide a medium which appears to be capable of dissolving a vast range of inorganic (and some organic) molecules to high concentrations, it was unexpected that two or more different ionic liquids each comprising materials dissolved therein, could be combined and yet allow the dissolved materials to remain stable in the solution. One would have expected one or both of the materials to precipitate out of the ionic liquid upon mixing. Thus, it was also unexpected that such a solution would yield a matrix having an agent homogenously distributed therein upon removal of most of the ionic liquid from the solution mixture.

The invention is illustrated further by the following non-limiting examples.

### Example 1

### Preparation of Neoproteoglycans

### Introduction:

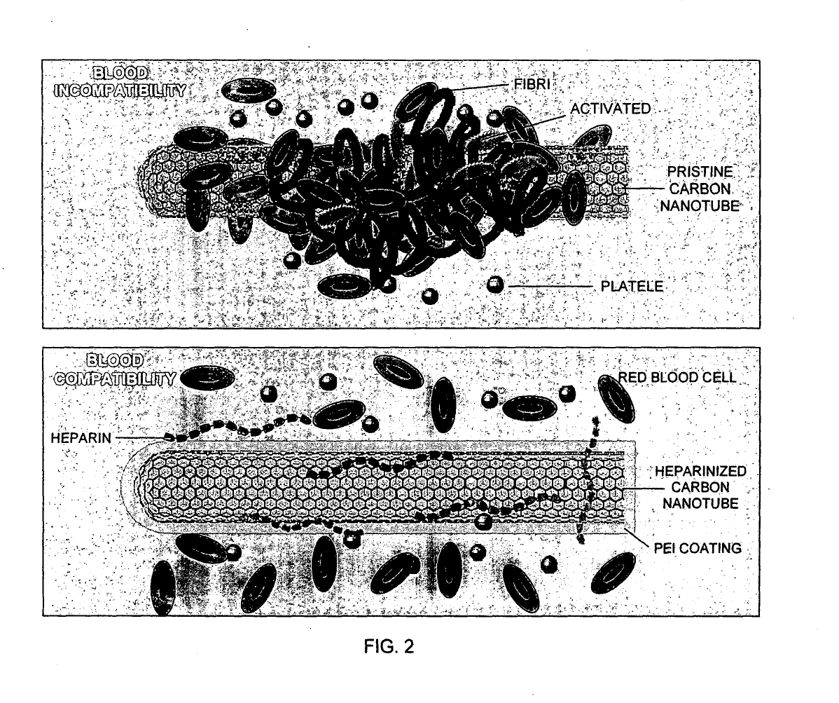

The present invention replaces the core protein of proteoglycans (PGs) with carbon nanotubes (CNTs) to afford highly stable neo-PGs for functional and structural studies. CNTs represent one of the most widely used building blocks for nanodevices and have also been successfully used as solid supports for biofunctionalization. CNTs with their unique structural, electronic and mechanical properties have an enormous number of applications in making various materials including nanotube polymeric composites, electronic and optical devices and enzyme/catalytic supports. CNTs, because of their high surface volume ratio, are also useful in making enzyme immobilized biosensors. Nanotubes are often preferred over metallic or non-metallic nanoparticles for biomedical applications because of their larger inner volume, distinct inner and outer surfaces and open mouths. These properties enable the filling of nanotubes with desired species (small molecule or macromolecule), differential modification of inner and outer surfaces and access to inner surfaces for incorporation of species. Multiwalled carbon nanotubes (MWNTs) with high stability and appropriate size (average diameter ˜40 nm) were selected in the current study to replace vulnerable core proteins. It was anticipated that heparinization of MWNTs would afford nanomaterials that could mimic endothelial heparan sulfate PGs. (FIG. 2) By following this approach, PEI-functionalized singlewalled carbon nanotubes, which showed potential as substrates for neuronal growth, can also be made blood compatible.

Three steps were used to prepare nano-based neoPGs: (a) coating of MWNTs with poly(ethyleneimine); (b) activation of tetrabutylammonium salt of heparin by cyanogen bromide²² and (c) coupling of activated heparin to MWNTs. Heparinized MWNTs were then characterized structurally using atomic force microscopy (AFM) and biologically using activated partial thromboplastin time (APTT) and Thromboelastography (TEG).

### Methods and Materials:

### Materials:

Sodium heparin from porcine intestinal mucosa was obtained from Celsius Laboratories. Multi walled carbon nanotubes (MWNTs) (average diameter 40 nm, length 10 μm) were obtained from Carbon Nanotechnology Inc. and used as is for the PEI coating. All other chemical used were obtained from Fisher Scientific. Human blood plasma and human whole blood used for the APTT and TEG studies were pooled samples obtained from healthy donors.

### Methods:

### (a) Poly(ethyleneimine) Coating of MWNTs:

MWNT (80 mg) was sonicated in 1% PEI aqueous solution for 3 h, followed by filtration using 0.8 μM polycarbonate filter and washed using double distilled water three times and dried in a dessicator to yield PEI coated MWNTs (PEI-MWNT).

### (b) Preparation and Activation of Tetrabutylammonium Salt of Heparin Using Cyanogen Bromide:

Heparin sodium salt from porcine intestinal mucosa (150 mg) was passed through a 30 ml column packed with cationic exchange resin (Dowex® H⁺ resin) to afford protonated heparin, which was then neutralized with 50% solution of tetrabutylammonium hydroxide in water to pH 7.0. The solution was then freeze dried to give TBA salt of heparin. The activation was done by using a previous protocol (ref 11 in the manuscript). Briefly, 100 mg of tetrabutylammonium heparin was dissolved in 1 ml of acetonitrile. 1 ml of 100 mg/ml of cyanogen bromide solution in acetonitrile was then added to the above solution in ice bath followed by the addition of 1.2 ml of 100 mg/ml solution of triethylamine in acetonitrile. The reaction contents became cloudy upon adding the base which then became a clear solution in a couple of minutes. Scheme 1 represents the schematic representation of this step.

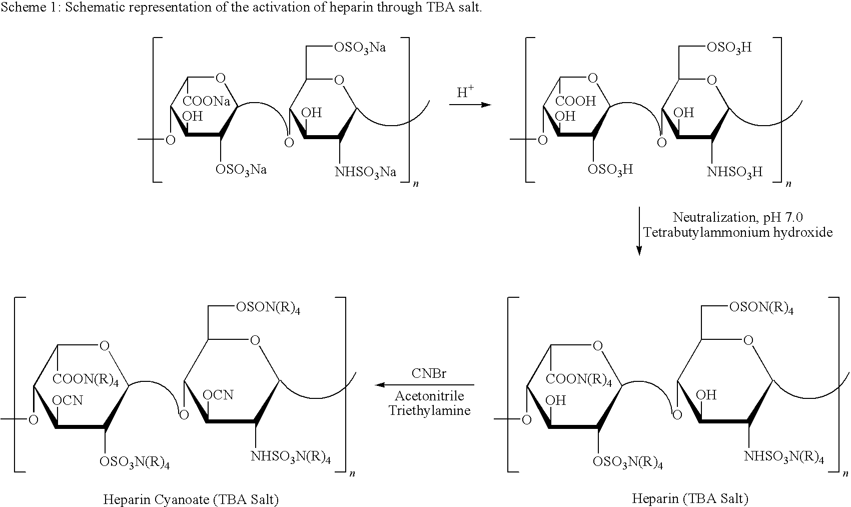

### (c) Immobilization of Activated Heparin Onto Nanotubes:

PEI coated nanotubes (20 mg) were suspended in 28.8 ml of 0.1 M sodium phosphate solution (pH 3.5) using sonication for 5 min. This solution was then added to the reaction solution prepared in step (b) (1:10 dilution of the solution made in step (c)). The resulting reaction mixture was then stirred for 2 h at room temperature followed by filtration using 0.2 μm polycarbonate filters. The heparinized nanotubes thus obtained were then washed with 25% saline solution for 15 min to remove the ionically and physically adsorbed heparin from the covalently formed heparinized nanotubes.

### (d) Characterization by Atomic Force Microscopy:

Heparinized MWNTs were characterized using Multimode IIIa atomic force microscopy (Digital Instruments/Veeco Metrology Group). Heparinized MWNTs were first suspended in DMF solution by sonication for 40 min and then spin-cast on silicon substrates. Tapping Mode (TM) AFM topography and phase images were recorded simultaneously in air. The driving frequency was adjusted to the resonant frequency (˜160 kHz) of a sharp probe (tip radius ˜2 nm, Mikromasch) scanned at a rate of 0.5027 Hz with 512 sample lines at a scale of 2 μm.

### (e) Carbazole Assay:

This assay looks for the presence of uronic acid (either as iduronic acid as in heparin or as glucuronic acid as in heparan sulfate) in a particular sample (ref 12 in the manuscript). Briefly, 1 mg of pristine MWNT was added to five test tubes (1-5), heparin in the amounts of 0 μg, 1 μg, 10 100 μg, 250 μg and 500 μg was added to test tubes 1-5 respectively. 1 mg of PEI-NT was added to test tube 6 and 1 mg of heparinized CNT was added to test tube 7. Test tube 8 was left without any MWNT as one of the controls. All the samples were then subjected to carbazole assay. The presence of heparin (precisely uronic acid) gives a pink color to the solution, the absorbance of which was then taken at 525 nm. The concentration of heparin loaded on to the MWNTs was found by using the standard obtained through the samples 1-5.

### (f) Activated Partial Thromboplastin Time (APTT):

This assay measures the prolonged clotting time as a function of heparin concentration. APTT is the time needed for plasma to form a clot after the addition of calcium and a phospholipid reagent such as activated cephaloplastin reagent. This assay is one of the available ways to determine the blood compatibility of a particular material. The protocol involves the addition of pristine MWNT (1 mg) or hep-MWNT (0.25 mg, 0.35 mg, 1.0 mg) into test tubes. 100 μl of citrated human plasma (platelet poor plasma) and 100 μl of automated APTT reagent were added to all the test tubes followed by incubation at 37° C. for 5 min. 100 μl of 0.025 M CaCl₂ was then added to recalcify the citrated blood plasma. The clotting time was measured by using automated Fibrometer which stops the timer as soon as the clot is formed.

### (g) Clotting Kinetics:

The clotting kinetics of the human whole blood was also assessed in the presence of the heparinized nanotubes by using thromboelastography (TEG). TEG has been a widely useful technique in hospitals to study the abnormalities in the coagulation pathway of the patients. TEG works by measuring the physical viscoelastic characteristics of blood. Typically, MWNTs (0.5 mg) was placed in a TEG cup, followed by the addition of 350 μl human whole blood and incubated for 5 min. 10 μl of 0.01 M CaCl₂ was added to recalcify the citrated blood. A coaxially suspended stationary piston was then placed on the cup with a clearance of 1 mm. This pin is suspended by a torsion wire which transduces the torque. The cup is oscillated at an angle of 4° 45′ in either direction every 4.5 s. During the clot formation, fibrin fibrils link the cup to the pin which influences the rotation of the pin, and the disturbance is measured and displayed by a computer. The display called thromboelastogram plots the torque experienced by the pin as a function of time. TEG studies the coagulation by measuring various factors including the latent time for clot initiation (R), the time to initiate a fixed clot firmness of around 20 mm amplitude (k), the kinetics of clot development (angle α) and the maximum amplitude (MA) of the clot. This is another way of measuring the blood compatibility of the heparinized nanotubes.

### (h) Lyase Digestion of Heparin Immobilized on MWNTs:

Heparinized nanotubes (1 mg) were treated with heparin lyase I (5 U (Sigma units)) in sodium phosphate buffer (1 ml, pH 7.1) at 37° C. for 24 h. After treatment, the heparinized nanotubes were washed with the same buffer, followed by drying in a dessicator.

### Results and Discussion:

While pristine MWNTs are hydrophobic, rapidly settling from water, coating with poly(ethyleneimine) (PEI) made them more hydrophilic affording solutions in water that are stable for more than two weeks. PEI also introduces free amino groups onto the MWNTs required for the immobilization of heparin. The amino groups that remain uncoupled after the immobilization of heparin were also used for the linking of a readily available fluorescent probe-fluorescein isothiocyanate (FITC) in stead of employing expensive fluorescent probes such as 1,1′,-dihexadecyl-3,3,3′,3′-tetramethylindocarbocyanine perchlorate or 3,3′-dihexyloxacarbocyanine iodide or BODIPY®, naphthalimides. Prakash et al., App. Phys. Lett. 2003; 83; 1219-1221 and Zhu et al., J. Mat. Chem. 2003; 13; 2196-2201. FITC remains stable on CNTs even after repeated washings with water and physiological buffers.

After the immobilization of heparin, heparinized MWNTs were extensively washed with 2M NaCl, followed by several times of water washes. All theses washes were analyzed for heparin by carbazole assay. Bitter and Muir, Anal. Biochem. 1962; 4; 330-334. The assay confirmed the absence of heparin in the later washes and the test medium, and demonstrated the stability of the heparinized MWNTs.

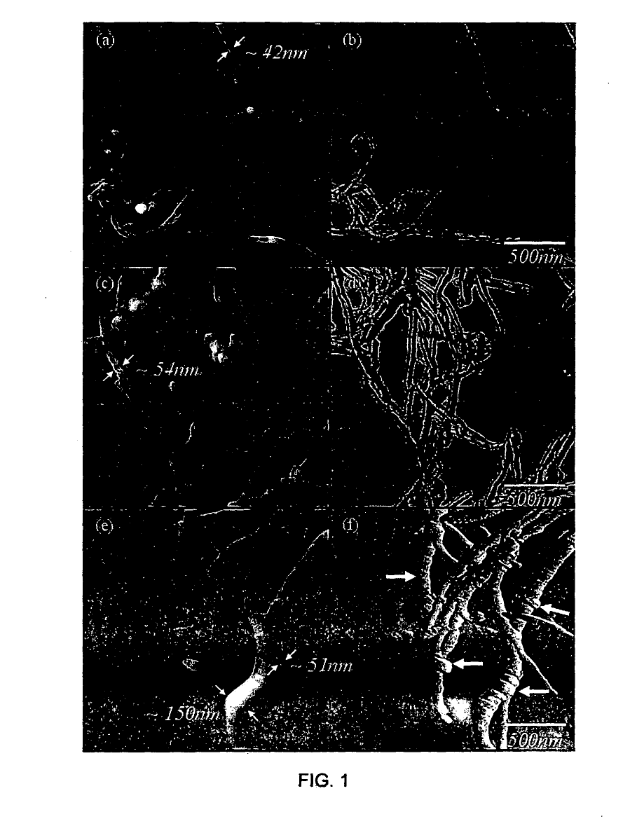

TM-AFM images of the pristine, PEI coated and heparinized MWNTs are shown in FIG. 1. Pristine nanotubes with an average diameter of 42 nm are shown in FIGS. 1 (a) and (b). Coating with PEI increased MWNT diameter by approximately 12 nm (FIGS. 1 (c) and (d)), and the covalent attachment of heparin produced conspicuous bulges on the MWNT surface increasing diameter by as much as 100 nm (FIGS. 1 (e) and (f)), suggesting that these bulges correspond to domains of immobilized heparin on the nanotube surface. Further, the PEI coating provided a homogenous increase in the nanotube diameter, while heparinization (a process of immobilization and not coating) led to a heterogeneous increase in the nanotube diameter. For instance, a PEI-coated nanotube without heparin immobilization (with diameter 51 nm) can also be seen along with heparinized nanotubes in FIG. 1 (e).

The amount of heparin present on the MWNTs was next determined chemically using carbazole assay. Bitter, T.; Muir, H. M. Anal. Biochem. 1962, 4, 330-334. The heparin loading on the MWNTs was 30% (w/w). The biological activity of heparin is most commonly determined by coagulation-based assays such as activated partial thromboplastin time (APTT), which measures the abnormalities in the intrinsic coagulation cascade of blood and thromboelastography (TEG), which measures the abnormalities in the global coagulation cascade of blood. Vance, N. G. Anesth. Analgesia. 2002, 95, 1503-1506. The APTT assay showed that pristine MWNTs did not prolong coagulation time while PEI coated MWNTs afforded only a small elongation of plasma based clotting time and only at a concentration above 1.5 mg/ml. This slight improvement in blood compatibility can be explained by the transformation of the hydrophobic surface of pristine MWNTs to a hydrophilic PEI coated surface. The APTT of heparinized MWNTs was significantly prolonged, giving linear dose response curve over the range of 0.75-3.0 mg/ml. Thus, heparin retains its expected plasma-based anticoagulant activity when immobilized on MWNTs.

Clotting kinetics experiments were next undertaken using whole human blood (Table 1). Again, pristine MWNTs showed a behavior similar to that of the control (no added MWNTs). PEI-coated MWNTs resulted in a slight prolongation of the clotting time (R) and a decrease in the clot strength (MA). Heparinized MWNTs (3 mg/ml) prolonged the clotting time by over 98 min and additional heparinized MWNTs fully anticoagulated the blood, resulting in its failure to clot over a 2 h period. Both the maximum clot strength and the rate of clot formation (a) observed with heparinized MWNTs were much lower than controls demonstrating their blood compatibility. The heparinized MWNTs were stable, remaining active even after extensive washing with 2 M sodium chloride. Treatment of heparinized MWNTs with heparin lyase 1 (Flavobacterial heparinase), nearly eliminated the anticoagulant activity, demonstrating that heparin was responsible for the observed anticoagulant activity and that the immobilized heparin chains were accessible to heparinase.

TABLE 1
TEG clotting kinetics of modified MWNTs.

| MWNTs | R (min) | K (min) | α (deg) | MA (mm) |
|---|---|---|---|---|
| Nil - control | 3.5 ± 0.3 | 3.1 ± 0.4 | 38.1 ± 2.2 | 39.5 ± 1.4 |
| Pristine MWNTs (1.5 mg/ml) | 3.8 ± 0.8 | 3.9 ± 1.1 | 49.4 ± 6.2 | 39.5 ± 3.5 |
| PEI-MWNTs (3.0 mg/ml) | 10.5 ± 3.1 | 15.9 ± 2.9 | 12.8 ± 2.4 | 27.7 ± 4.7 |
| Heparinized MWNTs (3.0 mg/ml) | 102.4 ± 10.1 | (a) | 0.5 ± 0.2 | 5.5 ± 2.3 |
| Heparinized MWNTs (6.0 mg/ml) | 133.9 ± 17.2^{(b)} | 0 | 0 | 0 |
| Lyase-treated heparinized | 11.9 ± 3.8 | (a) | 12.0 ± 3.6 | 19.9 ± 3.2 |
| MWNTs (1.5 mg/ml) | | | | |

[The clotting kinetics of the human whole blood was assessed in the presence of the heparinized nanotubes by using thromboelastography (TEG). R corresponds to the clotting time (min), k is the time to reach 20 mm clot amplitude, α is the rate of clot formation and MA is the maximum clot strength. The results are taken as an average of 3 experiments. (a) No clot of 20 mm amplitude was observed. ^{(b)}No clot observed.]

It is expected that this enabling technology would facilitate the making of nanodevices using these blood compatible nanomaterials as building blocks for biomedical applications such as artificial implants including structural tissue replacements i.e., artificial blood vessels or functional devices such as drug delivery matrices. Other GAGs such as chondroitin sulfate, hyaluronic acid can be similarly immobilized on the MWNTs to afford an array of nano-based neo-PGs having a wide range of potential biomedical applications, including structural elements in load bearing tissues such as cartilage.

### Conclusions

This report, being the first of its kind, introduced the ways of immobilizing heparin at nanoscopic dimensions. AFM studies showed the increase in the nanotube diameter confirming the immobilizing of heparin on the nanotube surface. APTT and TEG studies proved that heparin stays bioactive even in its immobilized form on the nanotube, rendering the nanotube blood compatibility.

### Example 2

### Ionic Liquid Derived Blood Compatible Composite

Introduction

A novel heparin and cellulose based biocomposite is fabricated by exploiting the enhanced dissolution of polysaccharides in room temperature ionic liquids (RTILs). This represents the first reported example of using a new class of solvents, RTILs, to fabricate blood compatible biomaterials. Using this approach, it is possible to fabricate the biomaterials in any forms such as films/membranes, fibers (nanometer or micron sized), spheres (nanometer or micron sized) or any shape using templates. A membrane film of this composite is evaluated as follows. Surface morphological studies on this biocomposite film showed the uniformly distributed presence of heparin throughout the cellulose matrix. Activated partial thromboplastin time and thromboelastography demonstrate that this composite is superior to other existing heparinized biomaterials in preventing clot formation in human blood plasma and in human whole blood. Membranes made of these composites allow the passage of urea while retaining albumin, representing a promising blood compatible biomaterial for renal dialysis, with a possibility of eliminating the systemic administration of heparin to the patients undergoing renal dialysis. Heparin immobilized to a surface, enhances the blood compatibility of that surface, reducing platelet adhesion, loss of blood cells, and increasing plasma recalcification time and activated partial thromboplastin time (APTT). Immobilized heparin, unlike soluble heparin, also inhibits initial contact activation enzymes through an antithrombin mediated pathway, and thus has enhanced anticoagulant properties.

Materials and Methods

Materials:

Heparin (sodium salt, extracted from porcine intestinal mucosa, USP activity 169 U/mg) was bought from Celsus Laboratories, Inc. Cellulose, [bmIm][Cl], [emIm][Cl] and Dowex® cationic resin were bought from Sigma Aldrich. Benzoic acid and imidazole was bought from Fisher Scientific. [EmIm] [ba] was prepared by following the existing protocol.²⁰

### Biocomposite Material Fabrication:

The imidazolium salt of heparin was prepared from the sodium salt using ion exchange chromatography (Dowex® cationic (H⁺) resin) followed by neutralization with imidazole. Approximately 7 mg of imidazolium heparin was added to 400 mg (500 μL) of [emIm][ba]. The contents were then mixed by vortexing and heated to 35° C. for about 20 min to afford a clear solution of heparin in [emIm][ba]. Cellulose was dissolved in [bmIm][Cl] (1.0 g) by preheating the RTIL to 70° C. and then adding 100 mg of cellulose. The contents were then mixed by vortexing and microwaved for 4-5 s to afford a 10% (w/w) cellulose in [bmIm][Cl]. The solution of heparin and cellulose in RTIL were combined and mixed by vortexing 5-10 s (other materials such as drugs or enzymes can be added at this step) to afford a solution of both polysaccharides. The resulting solution of heparin and cellulose can be fabricated into biomaterials in various shapes and forms including films/membranes (by membrane casting), micro- or nanospheres (atomization), micro- or nano-fibers (electrospinning) or any other shapes molded by using templates [FIG. 3]. Once solidified, this biocomposite was immersed in ethanol, which could dissolve both the RTILs leaving a composite of cellulose matrix with immobilized heparin, The biocomposite film was washed extensively with ethanol, to completely extract all RTIL (as confirmed by the absence of residual RTIL in the ethanol wash) and the biocomposite film was then dried in vacuo.

### Scanning Electron and Atomic Force Microscopic Techniques:

The cellulose and the heparin-cellulose composite films were analyzed with an electron beam at an acceleration voltage of 5 kV under Field Emission Scanning Electron Microscopy (FESEM) using a JEOL JSM-6332 equipped with secondary electron detectors. Prior to FESEM analysis, the film was subjected to gold sputtering to make the film conductive. The surface morphological differences between the cellulose film and a heparin-cellulose composite film were characterized using Tapping mode-atomic force microscopy (TM-AFM) on a Veeco D3100 Scanning Probe Microscope. The films were placed on an atomically flat silicon wafer surface and dried by applying weights in vacuo. Both the topography and the phase were recorded.

### Activated Partial Thromboplastin Time:

In a typical experiment, the film (0.5 cm×0.5 cm) was affixed to a cup of fibrometer (BBL Fibrometer, Beckton Dickinson Microbiology Systems, Cockeysville, Md.) followed by the addition of Automated APTT reagent (100 μl) and warmed for a minute at 37° C. Platelet-poor-human plasma (100 μl) was added, followed by 5 min incubation at 37° C., CaCl₂ (100 μl of 0.025 M) was then added to recalcify the citrated plasma. The plasma solution was observed for clotting by using the fibrometer. APTT was measured in triplicate.

### Thromboelastography

The clotting kinetics of the human whole blood was also assessed in the presence of the biocomposites by using thromboelastography (TEG). TEG has been a widely useful technique in hospitals to study the abnormalities in the coagulation pathway of the patients. TEG works by measuring the physical viscoelastic characteristics of blood. Typically, a biocomposite film (0.5×0.5 cm²) was placed in a TEG cup, followed by the addition of 350 μl human whole blood and incubated for 5 min. 10 μl of 0.01 M CaCl₂ was added to recalcify the citrated blood. A coaxially suspended stationary piston was then placed on the cup with a clearance of 1 mm. This pin is suspended by a torsion wire which transduces the torque. The cup is oscillated at an angle of 4° 45′ in either direction every 4.5 s. During the clot formation, fibrin fibrils link the cup to the pin which influences the rotation of the pin, and the disturbance is measured and displayed by a computer. The display called thromboelastogram plots the torque experienced by the pin as a function of time. TEG studies the coagulation by measuring various factors including the latent time for clot initiation (R), the time to initiate a fixed clot firmness of around 20 mm amplitude (k), the kinetics of clot development (angle α) and the maximum amplitude (MA) of the clot. This is another way of measuring the blood compatibility of the heparin-cellulose biocomposite films.

### Equilibrium Dialysis of Urea and BSA:

The film was fixed into an equilibrium dialysis cell between equal volumes of buffer. An 8 mg sample of either urea or BSA in PBS buffer (8 ml) was loaded onto the high-concentration-donor side (H) and eight ml of PBS buffer was loaded onto the low-concentration-receiver side (L). Aliquots were simultaneously removed at periodic intervals from each side, and their solute concentration was analyzed by measuring UV absorbance (206 nm for urea and 280 nm for BSA). Concentrations were calculated using a standard curve of each solute.

### Results and Discussion

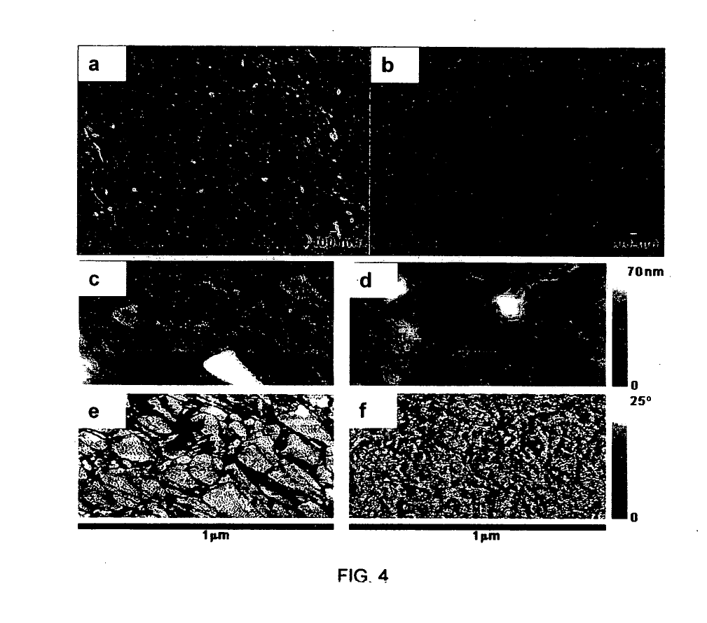

The FESEM micrographs of the cellulose and the heparin-cellulose composite films [FIG. 4 (a, b)] show uniformly formed nanosized pores throughout the films. This property suggested the utility as a membrane, particularly for dialysis applications. The heparin-cellulose composite film [FIG. 4 (b)] does not show the flaking observed in pure cellulose films [FIG. 4 (a)] and it has a uniform smooth surface with a larger number of nanopores. The nanopores found on the surface of this film have diameters ranging from 20-40 nm. The tapping mode AFM imaging [FIG. 4 (c)-(f)] clearly distinguished the cellulose and the heparin-cellulose composite films. The topography [FIG. 4 (c)] and the phase [FIG. 4 (e)] images of the cellulose film correlated to each other, both showing a monomodal distribution with a flaky structure in consistent with the FESEM observation. Phase imaging is sensitive to sudden changes in topography, such as at the edge of the flakes. However, the flakes themselves do not reveal any phase contrast indicating that they are of uniform elasticity, and composed of the same material (cellulose). In the case of heparin-cellulose composite film, the topographic image [FIG. 4 (d)] did not correlate with the phase image [FIG. 4 (f)]. The topography image (d) of heparin-cellulose composite film revealed the formation of globular shaped heparin domains in the cellulose matrix. This globular alignment was found uniformly across the surface. The phase image (f) reveals distinct bimodal contrast that does not correlate to topography. This contrast arises from difference in elasticity between the cellulose and heparin, indicating that there are two distinct phases (cellulose and heparin). This phase image (f) demonstrates heparin is uniformly distributed in the cellulose matrix.

The degree of entrapment of the water-soluble heparin by the water-insoluble cellulose was studied by incubating the composite film (105 mg) in water (10 ml). Samples of water were withdrawn at periodic time intervals and the heparin concentration was determined by carbazole assay against a standard curve. Bitter T, Muir H M. A modified uronic acid carbazole reaction. Anal. Biochem. 1962; 4:330-334. Residual heparin in the composite film could also be estimated by dissolving the composite film and subjecting this solution to the carbazole assay. The maximum leaching of heparin was observed in the first 20 min of the experiment, consistent with a commonly observed “burst effect”. Huang X, Brazel C S. “On the importance and mechanisms of burst release in matrix-controlled drug delivery systems”. J. Control. Release. 2001; 73:121-136 and Narasimhan B, Langer R. “Zero-order release of micro and macromolecules from polymeric devices: the role of burst effect”. J. Control. Release. 1997; 47:13-20. Little leaching was observed after this initial burst effect even after prolonged (100 h) washing, with less than 500 μg being lost from the 7 mg of heparin in the composite film. The results of carbazole assay used to determine the amount of heparin present before and after leaching of a heparin-cellulose composite suggest that 92% of the heparin within the heparin-cellulose composite film was not leachable. Further, elemental analysis (C, H, N, S) (Galbraith Laboratories, Knoxyille, Tenn., USA) of cellulose film showed no N or S while composite films showed the expected values based on the synthesis described in FIG. 1.

Alcian blue (a cationic dye that binds to the negative groups) staining clearly demonstrated the presence of dye accessible heparin even after leaching (data not shown) demonstrating that the heparin present within the composite was sufficiently accessible for chemical reaction.

Activated partial thromboplastin time (APTT) is routinely used for evaluating the blood compatibility of various heparinized polymer surfaces. Denizli A., J. App. Polym. Sci. 1999; 74:655-662 and Lin W C, Liu T Y, Yang M C., Biomaterials 2004; 25:1947-1957. APTT was used to measure the in vitro plasma anticoagulant activity of heparin present in both leached and unleached composite films against a cellulose control. Plasma on the unleached heparin-cellulose composite film did not clot as the film releases heparin into the plasma providing no measurable APTT. Plasma on cellulose behaved similar to control APTT of 32.3±2 s. Plasma on unleached composite showed no clot in over 1 h, while leached composite showed an APTT value of 2424.3±120.5 s (n=3)). This value compared favorably to other heparinized polymers including: poly(2-hydroxyethylmethacrylate)-dimethylaminoethylmethacrylate (P-(HEMA)-DMAEMA-HEP), (Denizili, supra) hexamethylene diisocyanate-tetraethylenepentamine-hexamethylene diisocyanate (HMDIO-TEP-HMDIC-HEP) [Marconi W, Benvenuti F, Piozzi A., Biomaterials 1997; 18:885-890], polyacrylonitrile-chitosan (PAN-C-HEP) [Yang M C, Lin W C., J. Polym. Res. 2002; 9:201-206] and poly(2-hydroxyethylmethacrylate) (P-(HEMA)-HEP) [Duncan A C, Boughner D, Campbell G, Wan W K., Eur. Polym. J. 2001; 37:1821-1826].

Thromboelastography (TEG) measures anticoagulant activity in human whole blood. Vance N G, Anesth. Analgesia 2002; 95:1503-1506. A comparison of thromboelastograms in the absence and presence of cellulose film reveals nearly identical curves. The thromboelastogram of the leached heparin-cellulose composite film shows an extended latent time of the clotting initiation. The shape of this thromboelastogram is also different from the controls demonstrating the presence of active anticoagulant heparin on the composite surface. The clotting parameters measured by TEG are given in Table 2.

TABLE 2

| Entity | R (min) | K (min) | α (deg) | MA (mm) |
|---|---|---|---|---|
| Human whole blood (control) | 3.9 | 1.7 | 65.6 | 55.9 |
| Plain cellulose film (control) | 4.6 | 1.7 | 65.9 | 55.9 |
| Heparin-cellulose composite | 23.0 | 7.8 | 20.4 | 44.8 |
| film-leached | | | | |
| Heparin-cellulose composite | 121.7^{(a)} | 0 | 0 | 0 |
| film-unleached | | | | |
| Free heparin (1 U/ml)³⁰ | 41.5 | 20 | 16.5 | 58.5 |

The time taken to form 20 mm clot (K) was prolonged, and the rate of clot formation (α) and the maximum clot strength (MA) were reduced when treated with leached heparin-cellulose composite film. Again, as with APTT, no clot was formed during the 2 h evaluation of the unleached composite and this was evaluated as owing to the release of weakly bound heparin. The clotting kinetics parameters of 1 U/ml solution heparin are also provided from the literature for comparison. Thus, the heparin-cellulose composite film has excellent blood compatible properties as observed by both plasma-based APTT (2424.3±120.5 s) and whole blood-based TEG studies.

The utility of the leached heparin-cellulose composite film for dialysis was evaluated next. The film was fixed into an equilibrium dialysis cell between equal volumes of buffer. The low MW (60.1) urea and the high MW (67,000) albumin involved in kidney dialysis were chosen for this equilibrium dialysis study across the composite film from high concentration side to low concentration side. An 8 mg sample of either urea or BSA in PBS buffer (8 ml) was added to the first compartment of the equilibrium dialysis cell, and the second compartment was filled with PBS buffer. Aliquots were taken at various time points from the second compartment, and the solute concentration was analyzed by measuring UV absorbance (206 nm for urea and 280 nm for BSA). Urea freely dialyzed across the membrane reaching equilibrium in less than 60 min. The diffusivity of urea across this membrane was found to be 3.6×10⁻¹⁰ m²/s. Only slight movement of BSA was seen across the membrane reaching <10% of its equilibrium value even after 45 h (data shown only for 5.6 h), suggesting a promising application for these heparin-cellulose composite films as kidney dialysis membranes.

Protamine sulfate, commonly used as a heparin reversal agent, can cause severe complications. The current approach avoids systemic heparinization, thus eliminating the need for protamine neutralization. Also during hemodialysis, on repeated exposure to heparin, patients can develop anti-heparin-platelet factor 4 antibodies.⁶ These antibodies are a major risk factors involved in many thrombotic complications including heparin-induced thrombocytopenia (HIT). The current approach should significantly reduce the concentration of circulating heparin. Hence, the chances for the generation of anti-heparin-platelet factor 4 antibodies and HIT may be significantly reduced.

In conclusion, the method of the invention has been used to fabricate a heparin-cellulose based biocomposite using RTILs. While this composite can be prepared in a variety of forms, preliminary studies evaluated a membrane film of this composite. This film was characterized using FESEM, AFM, dye binding, carbazole and elemental analysis. These analyses showed a very uniform composite in which heparin was stably immobilized but still chemically and biologically accessible. The biocompatibility of the film was clearly demonstrated by APTT and TEG measurements and implied superior performance to other heparinized biomaterials reported in the literature. Finally these membranes can be used in dialysis suggesting a potential new and valuable material to make blood compatible, hollow fiber, nanoporous membranes for kidney dialysis that might eliminate the requirement for systemic heparinization.

### Example 3

### Preparation of Blood Compatible Fibers by Electrospinning from Room Temperature Ionic Liquids

Introduction:

Electrospinning is a widely used simple technique to prepare micron to nanometer sized fibers of various polymers. Electrospun fibers find applications in the making of fiber-reinforced composites, membranes, biosensors, electronic and optical devices and as enzyme and catalytic supports. Electrospinning technique is useful even in large scale manufacturing environments such as textile industries. A variety of novel tissue engineering scaffolds have been prepared by electrospinning various synthetic and natural biodegradable polymers. However, the range of the polymers that can be electrospun is still limited by the availability of volatile solvents and their limited capability of dissolving polymers of different types. In this example, making electrospun fibers from a relatively novel solvent system—room temperature ionic liquids (RTILs) is described. RTILs have become more important in a wide array of chemical processes owing to their capability of dissolving both polar and non-polar compounds. Other desirable properties of RTILs include low or zero vapor pressure, low melting point, large liquidus range, high thermal stability, large electrochemical window and recyclability. Further, the properties of an RTIL can be modified by adjusting the structures of its anion or cation or both, and hence, RTILs are also called designer solvents. RTILs have proven to be a promising solvent system for the reactions involving biopolymers such as enzymes and carbohydrates. The successful application of RTILs in electrospinning could increase the number and types of materials from which the fibers can be made.

Electrospinning can be considered as a derivative of the electrospray process as both use high voltage to form a liquid jet. In the electrospinning process, a polymer solution is held by its surface tension at the end of a capillary. When a sufficiently large electric field is applied, the solution at the tip of the capillary elongates to form a cone due to coupled effects of the electrostatic repulsion within the charged droplet and attraction to a grounded electrode of opposite polarity. As the strength of the electric field is increased, the charge overcomes the surface tension, and a fine jet is ejected from the apex of the cone. Fibers were initially thought to be formed by the splitting of a primary jet into multiple filaments, a process known as “splaying”, but recent studies have shown that diameter reduction occurs due to the whipping action of a single jet as it nears the target. This whipping instability, caused by small lateral fluctuations in the centerline of the jet as it travels towards the target, causes high frequency bending and stretching of the jet, leading to the formation of micron and nanometer sized fibers. Typically, electrospinning involves the evaporation of solvent component of the visco-elastic liquid, resulting in fiber formation. In this report, we have demonstrated it is possible to electrospin cellulose and cellulose-heparin composite fibers from non-volatile room temperature ionic liquids (RTILs). Cellulose and heparin polysaccharides were selected as a model system also with potential applications as biomaterials. Since RTILs are low melting salts having very low vapor pressure, it is impossible to evaporate them. Instead, in this report, the RTIL is removed from cellulose and heparin by dissolution in ethanol co-solvent.

Cellulose, a linear polysaccharide composed of α (1→4) linked glucose, is known for its excellent biocompatibility, thermal and mechanical properties. The insolubility of cellulose in most conventional organic and aqueous solvents is attributed to its very high crystallinity supported by an extensive hydrogen bonding network. Cellulose fibers have been made by electrospinning from a variety of solvents such as acetone, acetic acid and dimethylacetamide. The RTIL, 1-butyl, 3-methylimidazolium chloride ([bmIm][Cl]) (FIG. 3) was reported to dissolve up to 25% (w/w) unmodified cellulose with the aid of microwave irradiation. Heparin is a linear, polydisperse, anionic polysaccharide that plays a vital role in regulating many biological activities. Heparin, the most widely used anticoagulant, has also been extensively investigated to prepare various blood-contacting polymer devices with good blood compatibility. Heparin is soluble only in a few organic solvents including dimethylformamide, dimethylsulfoxide and formamide. We have recently reported that the RTIL 1-ethyl, 3-methylimidazolium benzoate ([emIm][ba]), dissolves up to 2% (w/w) of the imidazolium salt of heparin.

Materials and Methods:

Preparation of RTIL solution. Imidazolium salt of heparin was prepared from the pharmaceutical sodium salt form (an extract from porcine intestinal mucosa, average molecular weight (MWavg)=12,500) by ion exchange chromatography (Dowex® cationic H⁺)resin) followed by neutralization with imidazole. Approximately 7 mg of imidazolium heparin was added to ˜400 mg of [emIm][ba] and mixed by vortexing and heated to 35° C. for about 20 min to afford a clear colorless solution. Using the protocol of Swatloski et al, cellulose (MWavg=5,800,000) was dissolved in the RTIL—[bmIm][Cl]. Swatloski, R. P.; Spear, S. K.; Holbrey, J. D.; Rogers, R. D. J. Am. Chem. Soc. 2002, 8, 4974-4975. Briefly, a 10% (w/w) cellulose solution was prepared by heating 1 g of [bmIm] [Cl] to 70° C., addition of 100 mg of cellulose, vortex mixing, and microwave irradiation for 4-5 s to afford a clear yellow solution. Both the RTIL solutions (10% (w/w) cellulose in [bmIm][Cl] and 2% (w/w) heparin in [emIm][ba]) were combined and mixed using a vortex for 2 min to afford a clear cellulose-heparin solution.

Electrospinning method. Both, the 10% (w/w) cellulose in [bmIm] [Cl] and the cellulose-heparin solution (prepared above) were subjected to electrospinning (FIG. 3). A 1 ml sample of polysaccharide RTIL solution was transferred to a syringe attached to a syringe pump and a voltage of 15-20 kV was applied to the needle of the syringe, with a grounded charge, in the form of an aluminum sheet placed beneath the ethanol collector. The nozzle to grounded target distance was fixed at 15 cm. The flow rate of the syringe pump (0.03-0.05 ml/min) was adjusted in tandem with the applied voltage affording fiber formation. Both of the RTILs selected for this study, [bmIm] [Cl] and [emIm] [ba] are completely miscible in ethanol while neither polysaccharides are ethanol soluble. Hence, as the fibers formed, the ethanol extractively removed the RTIL solvents affording pure polysaccharide fibers. The fibers, in the form of tangled web, were washed with additional ethanol, and then dried in vacuo to remove the residual ethanol.

Surface Characterization. A JEOL JSM-6332 FESEM equipped with secondary electron detectors was used at a voltage of 5 kV to study the surface characterization of the fibers. To perform the FESEM analysis, the fibers were first subjected to gold sputtering to form a monolayer of gold on the surface of the fiber to afford a conductive film.

Thromboelastography (TEG). The cellulose-heparin composite fibers were thoroughly washed with water to remove all leachable heparin prior to measuring TEG.²° Typically, dry fiber (1 mg) was placed in a TEG cup, followed by the addition of 350 μl human whole blood and incubated for 5 min. A 10 μA aliquot of 0.01 M CaCl₂ was added to recalcify the citrated blood. A coaxially suspended stationary piston was then placed in the cup with a clearance of 1 mm. This pin is suspended by a torsion wire which transduces the torque. The cup is oscillated at an angle of 4° 45′ in either direction every 4.5 s. During the clot formation, fibrin fibrils link the cup to the pin which influences the rotation of the pin, and the disturbance is measured and displayed by a computer. The display, called a thromboelastogram, plots the torque experienced by the pin as a function of time.

Results and Discussion:

A 10% (w/w) solution of cellulose dissolved in [bmIm][Cl], and another solution containing cellulose (in [bmIm][Cl]) and heparin (in [emIm][ba]) were prepared and subjected to electrospinning (FIG. 3). The fibers formed were directly received in ethanol that can completely dissolve both the RTILs used in the dissolution but neither of the polysaccharides are ethanol soluble. Hence, as the fibers formed, the ethanol extractively removed the RTIL solvents affording pure polysaccharide fibers. These fibers were then subjected to further washing with ethanol until there is no residual RTIL found by distillation. The fibers were then dried in vacuo to remove the residual ethanol. Both cellulose and cellulose-heparin composite fibers were made by this approach. Elemental analysis (C, H, N, S) (Galbraith Laboratories, TN) of cellulose fibers showed no N or S while cellulose-heparin composite fibers showed the expected values based on the synthesis described in FIG. 3, confirming the absence of RTILs in the dried fibers.

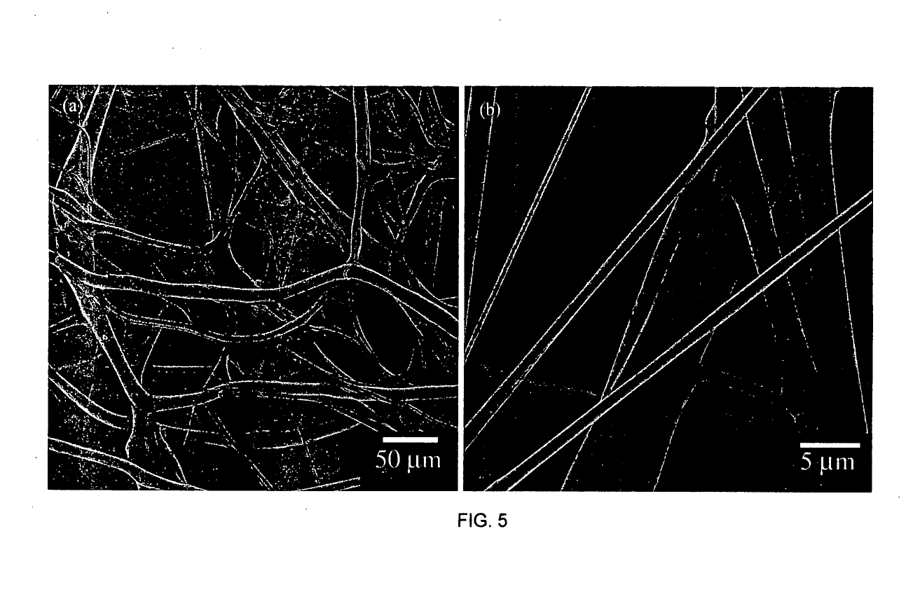

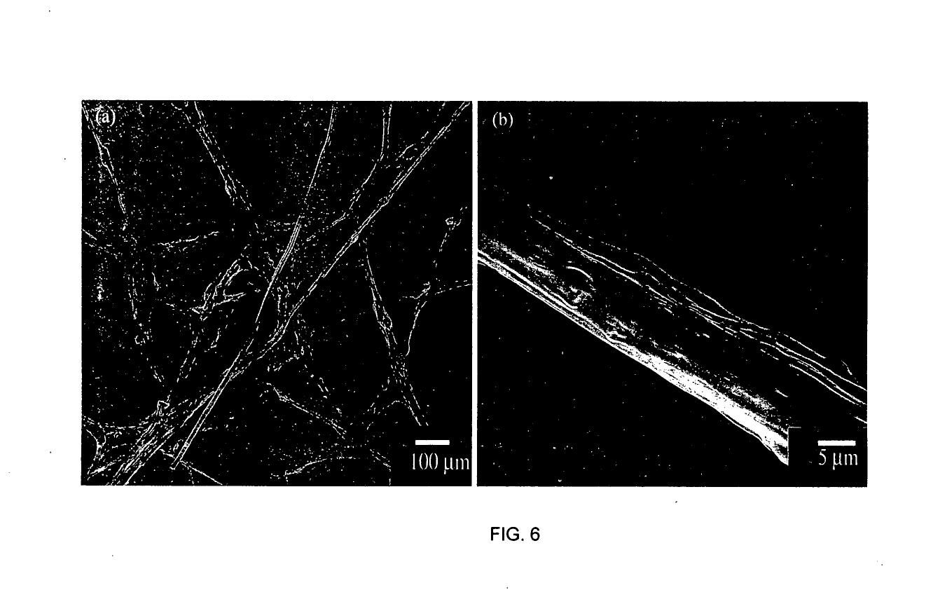

The dried cellulose and cellulose-heparin composite fibers were structurally characterized using field emission scanning electron microscopy (FESEM). The FESEM characterization of the cellulose only fibers (FIGS. 5 (a) and (b)) showed the formation of highly branched, nanometer to micron sized fibers by electrospinning from RTIL solutions. Cellulose fibers were made out of 10% (w/w) cellulose-RTIL solution. FESEM images of the cellulose-heparin composite fibers are shown in FIG. 6. The morphology and diameter distribution of electrospun fibers depend on a variety of process parameters, including the solution concentration, surface tension of solvent, applied voltage and solution feed rate. The high viscosity and non-volatility of the RTILs limited the fibers formed to mostly micron-sized diameters (see fiber diameter distributions in supplementary information), and also contributed to the interconnected branched structures. The mean fiber size for the cellulose/heparin composite was larger than that for pure cellulose, mainly due to the higher viscosity. Even when pure cellulose was electrospun from RTILs, only a small percent of nanoscale (˜500 nm) fibers were observed. By using low-viscosity RTILs and optimization of the spinning parameters, it should be possible to prepare non-branched nanofibers of cellulose/heparin composites. The surface roughness of the cellulose-heparin composite fibers was also much higher than that of the cellulose-only fibers (FIGS. 5 (b) and 6 (b)). This difference may be due to the phase separation of cellulose and heparin in the electrospinning process, although other phenomena such as the differential rate of solvent removal and skin formation due to differences in blend composition or the MW or fiber diameter might also contribute to the observed roughness of the composite fibers.

Biological characterization of the cellulose-heparin fibers was performed by measuring the clotting kinetics of human whole blood exposed to these fibers using thromboelastography (TEG). Mousa, S. A. et al., Asterioscler. Thromb. Vasc. Biol. 200; 20; 1162-1167. The clotting kinetics values of the human whole blood treated with the cellulose and the cellulose-heparin composite fibers are given in Table 3. In TEG studies, cellulose fibers behave similar to the control sample (no added fibers). In contrast, cellulose-heparin composite fibers afford a prolonged clotting time (R) in a concentration dependent fashion. The time taken to reach 20 mm of clot (K) increased and the rate of the clot formation (a) decreased. Little or no effect on maximum amplitude (MA) of clot formation was detected. These observations suggest the presence of heparin in an electrospun fiber acted as an anticoagulant slowing clot formation without altering the final amount of clot formed. It is noteworthy that heparin maintained its bioactivity even after an exposure to high voltages (10-20 kV) required in the electrospinning process.

TABLE 3
Clotting kinetics values of human whole blood treated
with fibers from RTILs

| Fibers | R (min) | K (min) | α (deg) | MA (mm) |
|---|---|---|---|---|
| Human whole blood | 3.8 | 2.6 | 61.9 | 50.0 |
| (control-no fibers) | | | | |
| Cellulose fibers | 4.8 | 1.6 | 63.9 | 55.7 |
| Cellulose-heparin fibers (1 mg) | 24.0 | 16.1 | 8.6 | 40.4 |
| Cellulose-heparin fibers | 69.4 | 43.3 | 5.1 | 50.8 |
| (1.8 mg) | | | | |

Conclusions

Cellulose and cellulose-heparin composite fibers have been made for the first time by electrospinning from RTILs. The use of RTILs to form fibers, followed by RTIL removal through ethanol extraction demonstrates an advantage for the high viscosity of RTIL solvents. FESEM images showed the formation of both micron and nanometer sized fibers. The cellulose fibers showed a smooth surface while the cellulose-heparin composite fibers had a rough surface morphology. Heparin, in spite of being a biological macromolecule, stayed intact and bioactive despite its exposure of high voltage during electrospinning. The application of RTIL solutions in fiber formation by electrospinning is expected to delimit the nature of polymer/material from which electrospun fibers can be made. Finally, cellulose-heparin fibers offer promise in the preparation of woven fabrics for use in the construction of artificial vessels with excellent blood compatibility.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. Blood compatible nanomaterials.

2. The nanomaterials of claim 1 wherein the nanomaterials comprises: carbon, polymeric compounds, block-polymeric compounds, non-polymeric compounds, metallic compounds, non-metallic compounds, or composites.

3. The nanomaterials of claim 1 in the shapes of hollow or solid, spheres, ellipsoids, fibers, wires, pyramids, prisms, tubes or any combination thereof.

4. The nanomaterials of claim 1 wherein the nanomaterials are in the shape of nanotubes.

5. The nanotubes of claim 4 wherein the nanotubes are single-walled nanotubes, double-walled nanotubes (DWNTs) or multi-walled nanotubes (MWNTs).

6. The nanomaterials of claim 1 comprising carbohydrates.

7. The nanomaterials of claim 1 comprising glycosaminoglycans.

8. The nanomaterials of claim 7 wherein the glycosaminoglycans are heparan sulfate, heparin, dermatan sulfate, keratan sulfate hyaluronate, chondroitin sulfate, derivatives thereof, hybrids thereof, or any combinations thereof.

9. The nanomaterials of claim 1 comprising a composite.

10. The nanomaterials of claim 9 wherein the composite comprises a modified or unmodified carbohydrate matrix having an agent homogenously distributed therein.

11. The nanomaterials of claim 10 wherein the agent is an anticoagulant, antithrombogenic, antibiotic, diagnostic, imaging agent, radioactive agent, fluorescent agent or anticancer agent.

12. The nanomaterials of claim 11 wherein the agent is heparan sulfate, heparin, dermatan sulfate, keratan sulfate hyaluronate, chondroitin sulfate, derivatives thereof, hybrids thereof, or any combinations thereof.

13. The nanomaterials of claim 10 wherein the matrix is modified or unmodified cellulose, hemicellulose, inulin, chitin, chitotsan, glycogen, starch, pectin, carageenan, fucan, or fucoidin.

14. The nanomaterials of claim 9 wherein the composite is in the form of a film, a membrane, a fiber, a sphere, a wire, a rod, an extruded shape, a molded shape, or any combination thereof.

15. A method of making a composite of claim 9 comprising the steps of:
(a) dissolving a matrix material in a first ionic liquid;
(b) dissolving at least one agent in at least one ionic liquid wherein at least one ionic liquid having an agent dissolved therein is different from the first ionic liquid of step (a);
(c) combining the first ionic liquid of step (a) with the at least one ionic liquid of step (b) and mixing to form a combined solution comprising the matrix material and at least one agent; and
(d) fabricating the combined solution of step (c) in the form of a composite nanomaterial.

16. The method of claim 15 further comprising the step of removing at least a portion of the residual ionic liquid after step (c) or step (d).

17. The method of claim 16 wherein the removing step comprises washing the solid composite with a cosolvent after step (d).

18. The method of claim 16 wherein the removing step comprises extracting the ionic liquid from the combined solution of (c) with a cosolvent.

19. The method of claim 18 wherein the removing step comprises extracting the ionic liquid from the composite after step (d).

20. The method of claim 17 wherein the cosolvent is ethanol, isopropanol, methanol, water, hexanes, ethyl acetate, or acetonitrile.

21. The method of claim 17 wherein one or more of the ionic liquid are regenerated from the cosolvent by distillation.

22. The method of claim 15 further comprising drying the composite nanomaterial.

23. The method of claim 15 wherein the combining step is conducted at temperatures and under conditions in which the matrix and the agent are both stable.

24. The method of claim 15 wherein step (b) comprises at least two agents dissolved in at least two separate and different ionic liquids.

25. The method of claim 15 wherein step (b) comprises at least two agents dissolved in the same ionic liquid.

26. The method of claim 15 wherein the matrix is modified or unmodified cellulose, hemicellulose, inulin, chitin, chitotsan, glycogen, starch, pectin, carageenan, fucan, or fucoidin.

27. The method of claim 15 wherein the agent is heparan sulfate, heparin, dermatan sulfate, keratan sulfate hyaluronate, chondroitin sulfate, derivatives thereof, hybrids thereof, or any combinations thereof.

28. The method of claim 15 wherein the ionic liquids of step (a) or step (b) are selected from: 1-butyl, 3-methylimidazolium chloride [bmIm][Cl], 1-ethyl, 3-methylimidazolium benzoate ([emIm][ba]), 1-butyl, 3-methylimidazolium benzoate ([bmIm][ba]), and 1-butyl, 3-methylimidazolium hexafluorophosphate [bmIm][PF₆].

29. The method of claim 15 wherein the fabricating step comprises, casting to form molded shapes, films and membranes, electrospinning to provide fibers, or atomizing to form spheres and particles having smooth or textured surfaces.

30. The method of claim 26 wherein the matrix is cellulose and wherein the cellulose concentration in the first ionic liquid is between about 1% (w/w) and 50% (w/w).

31. The method of claim 30 wherein the agent is heparin and wherein the heparin concentration in the ionic liquid is between about 0.001 mg/mL and 0.5 mg/mL.

32. A biomaterial comprising the nanomaterials of claim 1.

33. A medical device comprising the nanomaterials of claim 1.

34. The medical device of claim 33 selected from the group consisting of: intravascular medical devices; extravascular medical devices, drug delivery devices, gene delivery devices, cell growth matrices, and any components thereof.

35. A composite comprising heparin homogenously distributed within a cellulose matrix.

36. The composite of claim 35 in the form of a nanomaterial.

37. The composite of claim 35 in the form of a fiber selected from hollow or solid nanofibers, microfibers, or macrofibers.

38. The composite of claim 37 wherein said fibers are fabricated to form a biomaterial.

39. The composite of claim 35 wherein the biomaterial is fabricated to form a blood compatible medical device for renal dialysis.

40. A method of treating a patient in need of renal dialysis comprising incorporating the medical device of claim 36 into the dialysis process.

41. The method of claim 40 wherein the patient has not been treated systemically with an anticoagulant prior to, or during dialysis.

42. A neoglycoprotein comprising at least one carbohydrate linked to a nanomaterial core.

43. The neoglycoprotein of claim 42 wherein the nanomaterial core comprises carbon, polymeric compounds, block-polymeric compounds, non-polymeric compounds, metallic compounds, non-metallic compounds, or composites spheres, full spheres, ellipsoids, fibers, wires, pyramids, prisms, tubes or any combination thereof.

44. The neoglycoprotein of claim 42 wherein the nanomaterial core comprises nanotubes.

45. The neoglycoprotein of claim 42 wherein the nanotubes are comprised of carbon.

46. The neoglycoprotein of claim 45 wherein the nanotubes are single-walled nanotubes (SWNTs), double-walled nanotubes (DWNTs) or multi-walled nanotubes MWNTs).

47. The neoglycoprotein of claim 42 wherein the carbohydrate is modified or unmodified cellulose, hemicellulose, inulin, chitin, chitotsan, glycogen, starch, pectin, carageenan, fucan, or fucoidin.

48. The method of claim 42 wherein the neoglycoprotein is blood compatible.

49. The method of claim 42 wherein the neoglycoprotein is a neoproteoglycan.

50. A solution comprising a mixture of a matrix material dissolved in a first ionic liquid in combination with at least one agent dissolved in at least one different ionic liquid wherein the matrix and the agent remain stable in the solution mixture.

51. The solution of claim 50 wherein the matrix material is cellulose and the agent is heparin.

52. The solution of claim 51 wherein upon removal of the ionic liquid, the solution mixture forms a composite having a cellulose matrix with heparin homogenously distributed therein.

53. The solution of claim 50 wherein upon removal of the ionic liquid, the solution mixture forms a composite comprising the matrix with the agent homogenously distributed therein.

54. A method of making a composite comprising the steps of:
(a) dissolving a matrix material in a first ionic liquid;
(b) dissolving at least one agent in at least one ionic liquid wherein at least one ionic liquid having an agent dissolved therein is different from the first ionic liquid of step (a);
(c) combining the first ionic liquid of step (a) with the at least one ionic liquid of step (b) and mixing to form a combined solution comprising the matrix material and at least one agent; and
(d) fabricating the combined solution of step (c) in the form of a composite nanomaterial.

55. The method of claim 54 further comprising the step of removing at least a portion of the residual ionic liquid after step (c) or step (d).

56. The method of claim 55 wherein the removing step comprises washing the solid composite with a cosolvent after step (d).

57. The method of claim 55 wherein the removing step comprises extracting the ionic liquid from the combined solution of (c) with a cosolvent.

58. The method of claim 55 wherein the removing step comprises extracting the ionic liquid from the composite after step (d).

59. The method of claim 55 wherein the removing step requires a cosolvent and the cosolvent is ethanol, isopropanol, methanol, water, hexanes, ethyl acetate, or acetonitrile.

60. The method of claim 59 wherein one or more of the ionic liquids are regenerated from the cosolvent by distillation.

61. The method of claim 54 further comprising drying the composite nanomaterial.

62. The method of claim 54 wherein the combining step is conducted at temperatures and under conditions in which the matrix and the agent are both stable.

63. The method of claim 54 wherein step (b) comprises at least two agents dissolved in at least two separate and different ionic liquids.

64. The method of claim 54 wherein step (b) comprises at least two agents dissolved in the same ionic liquid.

65. The method of claim 54 wherein the matrix is modified or unmodified cellulose, hemicellulose, inulin, chitin, chitotsan, glycogen, starch, pectin, carageenan, fucan, or fucoidin.

66. The method of claim 54 wherein the agent is heparan sulfate, heparin, dermatan sulfate, keratan sulfate hyaluronate, chondroitin sulfate, derivatives thereof, hybrids thereof, or any combinations thereof.

67. The method of claim 54 wherein the ionic liquids of step (a) or step (b) are selected from: 1-butyl, 3-methylimidazolium chloride [bmIm][Cl], 1-ethyl, 3-methylimidazolium benzoate ([emIm][ba]), 1-butyl, 3-methylimidazolium benzoate ([bmIm][ba]), and 1-butyl, 3-methylimidazolium hexafluorophosphate [bmIm][PF₆].

68. The method of claim 54 wherein the fabricating step comprises, casting to form molded shapes, films and membranes, electrospinning to provide fibers, or atomizing to form spheres and particles having smooth or textured surfaces.

69. The method of claim 65 wherein the matrix is cellulose and wherein the cellulose concentration in the first ionic liquid is between about 1% (w/w) and 50% (w/w).

70. The method of claim 66 wherein the agent is heparin and wherein the heparin concentration in the ionic liquid is between about 0.001 mg/mL and 0.5 mg/mL.

71. The method of claim 54 wherein the agent is an anticoagulant, antithrombogenic, antibiotic, diagnostic agent, imaging agent, radioactive agent, fluorescent agent or anticancer agent.

72. A composite prepared in accordance with the method of claim 54.

73. The composite of claim 72 wherein the composite is a nano-sized material, a microsized material or a macro-sized material.
